(19) United States
(12) Patent Application Publication
(10) Pub. No.: US 2026/0325088 A1
(43) Pub. Date: Oct. 22, 2026

(54) DEVICE AND METHOD FOR GENERATING A RADIOLABELED DRY AEROSOL

(71) Applicant: AlmediS Altmann GmbH, Salzgitter (DE)

(72) Inventors: Björn Altmann, Salzgitter (DE); Andreas Reparon, Salzgitter (DE); Reiner Paul, Salzgitter (DE); Bernd Altmann, Salzgitter (DE); Werner Schwarz, Salzgitter (DE)

(73) Assignee: AlmediS Altmann GmbH, Salzgitter (DE)

(21) Appl. No.: 19/119,527

(22) PCT Filed: Oct. 13, 2023
(86) PCT No.: PCT/EP2023/078528

§ 371 Date: Apr. 9, 2025

(30) Foreign Application Priority Data
Oct. 14, 2022 (DE) 10 2022 127 030.0

(51) Int. Cl. A61B 6/50; A61B 5/091; A61B 5/097; A61B 6/40; A61K 51/12

(52) U.S. Cl. CPC A61B 6/50; A61B 5/091; A61B 5/097; A61B 6/4057; A61K 51/1206; A61K 51/1244

(57) ABSTRACT

An apparatus and method are provided which ensure fast, safe and reproducible production of a radioactively labeled dry aerosol. This dry aerosol enables reproducible imaging of the alveolar space. The apparatus includes a probe for measuring the oxygen content in the combustion chamber, a vacuum pump, an air compressor, a camera, a gamma counter, a flow meter that is configured to determine a patient's respiratory volume, at least one battery and/or PowerCap/buffer capacitor and/or high-performance capacitor, an inverter, an intelligent power electronics unit configured to analyze the sine wave of AC voltage of the grid current, and an external radioactivity sensor connected to the apparatus and configured to detect the increase in radioactivity in the patient's lungs upon administration of the produced radioactive dry aerosol.

The invention relates to an apparatus and a method for generating a radioactively labeled dry aerosol. The invention preferably belongs to the technical field of apparatuses and methods for conducting medically relevant measurements, in particular for diagnostic procedures in connection with the examination of lung function and the diagnosis of pulmonary embolisms.

### BACKGROUND AND PRIOR ART

The use of radioactively labeled materials to analyze biological and non-biological processes is known from the prior art. In particular, lung scintigraphy can be used to examine the ventilation of the lungs. This examination is often combined with an examination of the perfusion in the lungs. A radioactively labeled aerosol, for example of technetium particles, is inhaled into the lungs to examine ventilation in particular. The diffusion of the inhaled aerosol in the lungs is monitored using a gamma camera. If this examination reveals areas of the lungs that are poorly perfused but are ventilated, a pulmonary embolism can be diagnosed.

An apparatus for generating aerosols is used to produce the radioactively labeled aerosol. One existing example of such an apparatus is called the Technegas Generator. A technetium eluate is filled into a carbon crucible, which is heated to generate the aerosol. However, the heating process is difficult to control, meaning that the quantities of aerosol produced are often not reproducible. It has also been found that the energy required to heat the carbon crucibles to a sufficiently high temperature can cause power interruptions in the electricity grids. The available power supply also strongly influences the operation of the known apparatus for producing the aerosol.

The production of the dry aerosol has the strict requirement that it needs a peak power of well over 3000 watts for seconds. In order to achieve firing (white-hot heating) of the carbon crucible at a high firing temperature in the shortest possible heating time, a peak power of well over 3000 watts is required in the first second of heating. This requires a stable power supply at the user's premises, which is not available everywhere. A known problem with the Technegas Generator is that the fuse in the medical practice's power distribution box can trip and switch off due to the very high amount of energy required in the short period of time. Such a power cut-off can affect other devices or equipment in the practice and is therefore dangerous.

It is also important that the dose of radioactive particles delivered to the patient during the ventilation examination is sufficient but limited. This is because too low a concentration reduces the quality of the subsequently generated gamma camera images, while too high a concentration may preclude the performance of a subsequent examination of the perfusion with another radioactively labeled material, which is necessary during pulmonary embolism diagnostics. One reason for this is that the total amount of radioactivity that can be administered to the patient is limited.

With known apparatuses, the rate at which the carbon crucible is heated and the rate of release of the dry aerosol varies greatly from apparatus to apparatus and from application to application. As a result, the quality and concentration of the dry aerosol also varies in unpredictable ways. Currently, there is no known apparatus or method for producing a radioactively labeled aerosol of consistent quality. In particular, there is a need to reduce the yield variation, i.e. the difference in the efficiency of the aerosol production process, between apparatuses and between uses of the same apparatus.

One method that has been considered is to reduce the thickness of a protective glass that shields the open combustion chamber from the user. This allows better visibility when filling with the eluate of the radioactive material such that it can be injected into the carbon crucible with greater precision. However, this reduces the protection of the user, who may be a doctor, nurse or technician repeatedly introducing radioactive material into the apparatus. There is therefore a need for a solution that ensures precise insertion and positioning of the correct amount of eluate while providing a high level of safety for the user.

In addition, the speed at which the radioactively labeled aerosol can be produced and the concentration of radioactively labeled particles is currently very limited. The known apparatuses require up to 2 seconds to heat the carbon crucible to the required temperature for the release of a dry aerosol. However, this heating time can vary greatly in length, resulting in a level of deviation that makes it difficult to achieve reproducibility in the production of the dry aerosol. A further aim is therefore to greatly reduce the occurrence of such deviation. The heating time is crucial for the quality and particle quantity of the dry aerosol. A long heating time increases the number of breaths a patient has to take in order to perform the lung scintigraphy. It is therefore also necessary to produce the aerosol at a consistently high speed and concentration.

Furthermore, currently known apparatuses do not provide any procedure for ensuring that the examination of a patient's ventilation using a radioactive label is safe and appropriate. This is left to the expertise of the user. In reality,
however, users have varying levels of experience and may not be able to assess the suitability of a diagnostic procedure for a particular patient. There is therefore a need for a solution that allows an objective assessment of a patient's suitability for inhalation of the radioactively labeled aerosol before it is administered.

The existing technology for producing a radioactively labeled aerosol, in particular for diagnostic purposes, is therefore in great need of improvement.

### OBJECTIVE OF THE INVENTION

One objective of the invention was to provide an apparatus and a method which ensure fast, safe and repeatable production of a radioactively labeled dry aerosol. A further objective of the invention was to provide an apparatus and a method which enable fast, safe and repeatable imaging of the alveolar space. Further objectives of the invention include providing a system comprising various disposable components in addition to an apparatus for the fast, safe and repeatable production of a radiolabeled dry aerosol.

### DESCRIPTION OF THE INVENTION AND PREFERRED EMBODIMENTS

The objective is solved by the features of the independent claims. Advantageous embodiments of the invention are disclosed in the dependent claims and in the description.

In a first aspect, the invention relates to an apparatus for producing a radioactively labeled dry aerosol comprising
a carbon crucible with an open cavity suitable for filling with radioactive nuclide,
a combustion chamber which is suitable for holding a carbon crucible,
a current pulse generator which enables flash heating of the carbon crucible and a radioactive nuclide contained therein to form a dry aerosol of radioactively labeled carbon particles which can then be passed from the apparatus into the lungs of a patient for medical imaging, and
a control unit.

The filling volume of the carbon crucible is 0.1 mL-1 mL, preferably at least 0.2 mL, at least 0.4 mL, at least 0.5 mL or at least 0.6 mL. Preferably, the filling volume of the carbon crucible is 0.3-0.6 mL and particularly preferably 0.4-0.5 mL. Furthermore, the apparatus comprises:
a probe for measuring the oxygen content in the combustion chamber,
a vacuum pump for venting the combustion chamber,
an air compressor to ventilate the combustion chamber,
a camera that enables the filling of the carbon crucible to be monitored,
a gamma counter which is suitable for measuring a change in the amount of radioactivity in the carbon crucible and/or the combustion chamber,
a flow meter that is configured to determine a patient's respiratory volume,
at least one battery and/or PowerCap/buffer capacitor and/or high-performance capacitor,
an inverter for transforming the direct current of a battery and/or a PowerCap/buffer capacitor and/or a high-performance capacitor into an alternating voltage sine wave,
an intelligent power electronics unit for power grid operation of the apparatus with transformer, which is configured to analyze the sine wave of the alternating voltage of the mains current, wherein the sine wave preferably exhibits a standard frequency of 50-60 Hz and the alternating voltage of the mains current is preferably 100-400 volts, in particular 110 to 260 volts, even more preferably 230 volts,
an external radioactivity sensor connected to the apparatus and configured to detect the increase in radioactivity in the patient's lungs upon administration of the produced radioactive dry aerosol,
optionally an apparatus for integrating an inert or shielding gas source,
optionally an external ventilation bag which can be connected to the apparatus and is configured to increase the volume of radioactively labeled dry aerosol inhaled by a patient.

In the context of the invention, a “firing” of the carbon crucible is preferably a white-hot heating of its material. During such firing, carbon particles are preferably split off from the surface of the crucible. Preferably, the carbon is partially vaporized and sublimated during this process. The radioactive material (e.g. dried technetium) on the surface of the crucible is preferably also sublimated.

In the context of the invention, a “dry aerosol” is preferably a suspension of fine solid particles in air or another gas, the fine solid particles preferably having a diameter of less than 1 μm. By way of illustration, smoke may be considered as an example of a dry aerosol. The dry aerosol produced by the invention comprises preferably fine particles of a radioactive nuclide such as technetium or gallium and carbon particles in air, an inert gas or a mixture thereof.

In the context of the invention, a “nuclide” is preferably an atomic nucleus which is characterized by a certain number of protons and neutrons.

In the context of the invention, a “PowerCap” is preferably a power capacitor. This is preferably an electrical device that can store and discharge electrical energy. In principle, this preferably comprises one or more pairs of plates separated by an insulating material, the plates being attached to two terminals which allow the stored energy to be discharged into a circuit when required. A PowerCap preferably smooths the flow of current through the apparatus, reducing fluctuations that could trip a fuse or damage electrical components.

In the context of the invention, a “current pulse” is preferably an electric current that exhibits pulse sequences. Preferably, the pulse current consists of short pulses separated by a longer pause without current flow. Such a current can be emitted by an inverter, for example. The person skilled in the art is aware of other mechanisms that can serve as “current pulse generators” within the context of the invention.

In an embodiment of the apparatus, the inert or shielding gas is selected from argon, helium, acetylene and gas mixtures thereof, nitrogen and carbon dioxide. In a preferred embodiment of the apparatus, the inert or shielding gas is argon.

In a preferred embodiment of the apparatus, the radioactive nuclide is selected from technetium (Tc-99m), gallium-68, molybdenum-99, chromium-51, holmium-166, rubidium-82, iron-59, lutetium-177, palladium-103, potassium-42, scandium-47, selenium-75, sodium-24, xenon-133, ytterbium-169, ytterbium-177, iodine-131, iodine-125, samarium-153, rhenium-186, lutetium-177, phosphorus-32 or caesium-131, wherein technetium (Tc-99m) and gallium-68 are particularly preferred.

In one embodiment, the nuclide is filled into the carbon crucible as an eluate.

The selection of a suitable nuclide depends on various factors. With gallium 68, it is possible to perform a lung ventilation examination with PET camera imaging. PET cameras have a higher resolution than gamma cameras, which can be used with technetium. Technetium, however, has an energy of 133 keV, while gallium has 511 keV. This means that gallium is significantly more radiation-intensive and therefore requires greater shielding to protect the user.

In a further preferred embodiment of the apparatus, the carbon crucible comprises graphite. Preferably, graphite has a very high degree of purity, which is at least 97 wt. %, at least 98 wt. %, at least 99 wt. %, at least 99.7 wt. % or most preferably at least 99.9 wt. %. The carbon crucible can, for example, be an “Almedis High-Volume Crucible”. Preferably, the carbon crucible has a weight of 0.8-1.6 g, particularly preferably 1.15-1.25 g, in particular approx. 1.2 g. A preferred volume of the carbon crucible is at least 0.1 mL, preferably at least 0.2 mL, more preferably at least 0.3 mL, particularly preferably at least 0.4 mL. A preferred volume of the carbon crucible is at most 1 mL, preferably at most 0.8 mL, more preferably at most 0.6 mL.

In a preferred embodiment of the invention, the apparatus comprises electrical contacts which engage with the carbon crucible in order to heat it. Preferably, the electrical contacts comprise a material which is conductive and which can withstand both high temperatures and high currents. Preferably, the electrical contacts are carbon contacts. In a further embodiment, the contacts are embedded in a metal block, wherein the metal may be selected from brass, copper or aluminum. The contacts can be interchangeable contacts, which must be replaced after a predetermined number of uses. For example, the interchangeable contacts are removed from the combustion chamber with a wrench and replaced after every 50 firings (50 times the crucible is heated).

In a further preferred embodiment of the apparatus, the device does not comprise interchangeable contacts that have to be replaced by the user. Instead, the contacts can be long-term contacts, preferably made of carbon. Such contacts only need to be replaced after 500 firings, for example. This replacement can be carried out by a technician during maintenance of the entire apparatus. Regular replacement of the contacts at short intervals is no longer necessary.

In a further preferred embodiment of the apparatus, the radioactively labeled dry aerosol comprises nanoparticles, wherein the nanoparticles preferably exhibit an average diameter between 5-250 nm, preferably 10-100 nm, more preferably 30-60 nm, particularly preferably between 40-50 nm. Preferably at least 60%, preferably at least 75%, in particular about 80% of the nanoparticles exhibit a diameter of less than 100 nm.

In a further preferred embodiment, the apparatus comprises means for automatically bringing the carbon crucible into contact with electrical contacts, wherein the means are preferably also configured for automatic “looping” between the crucible and the contacts. The electrical contacts are preferably configured to pass current through the carbon crucible, which can be placed between them, such that the carbon crucible acts as a heating element.

Preferably, the electrical contacts comprise a material that can withstand both high temperatures, in particular up to 3000° C., and high currents. The electrical contacts are particularly preferably made of carbon. The electrical contacts are preferably embedded in a mounting block. Preferably, the mounting block comprises a material that can also withstand high temperatures and high currents. Particularly preferably, the mounting block comprises a metal or a metallic alloy, preferably brass, aluminum or copper.

The apparatus preferably comprises two electrical contacts in two mounting blocks. The mounting blocks are preferably connected to electrical cables of an electrical circuit.

The apparatus is preferably used to produce a dry aerosol, in particular from ultrafine carbon particles. The carbon particles are preferably “labeled” with the radioactive nuclide. Preferably, the particles of the dry aerosol comprise hexagonal platelets of carbon. Preferably, the radioactive nuclide attaches to the hexagonal platelets in order to label them. Preferably, an average ratio of the layer thickness to the diameter of the hexagonal platelets is 1:20-1:5, in particular approx. 1:10. This dry aerosol is advantageously suitable for inhalation, in particular for recording the lung function of a patient with a gamma camera and/or PET camera. This can be used, for example, to quantify a pulmonary embolism, possibly the severity of an acute or chronic obstructive airway disease and/or possibly also to diagnose the degree of obstruction in Covid 19 patients. Furthermore, the dry aerosol can be used in postoperative examinations, in particular in lung volume reductions. In a preferred embodiment, the dry aerosol can be used in preoperative and postoperative examinations.

By means of the apparatus, the carbon crucible can be heated to 2500° C.-3000° C., in particular approx. 2750° C., in less than 2 seconds, preferably up to 1.8 seconds, preferably up to 1.5 seconds, preferably up to 1.2 seconds, preferably up to 1 second, preferably up to 0.8 seconds, even more preferably up to 0.6 seconds and particularly preferably up to 0.5 seconds. This is preferably done in an oxygen-free atmosphere. In the context of the invention, this period is preferably referred to as “risetime”. After the risetime, the temperature (also referred to as “firing temperature” in the context of the invention) of the carbon crucible is maintained at 2500° C.-3000° C., in particular approx. 2750° C., for 10-20 seconds. Preferably, the carbon crucible is held at approx. 2750° C. for 13-17 seconds, in particular for approx. 15 seconds. The dry aerosol is preferably produced during the risetime and the period of maintained heating. The energy for raising and maintaining the temperature of the carbon crucible is preferably provided by mains current or by battery operation. It may be preferable for the carbon crucible to be supplied with mains current when it is heated. In this case, it may be advantageous to use a triac and/or transformer to regulate the electrical current and/or voltage supplied to the carbon crucible. It may also be preferred that the heating of the carbon crucible is battery powered. In this case, it may be advantageous to use zero-crossing pulse width modulation to regulate the current supplied to the carbon crucible. It is particularly preferred that an energy storage unit is used for heating. For example, batteries, PowerCaps and/or high-performance capacitors can be used. In this way, the short-term energy requirement can be adequately covered without creating instability in a network. Potentially dangerous power failures can be avoided. In particular, integrated batteries can enable a stable power supply to the carbon crucible during the firing process, in particular to enable a permanent reduction in risetime to less than 2 seconds, preferably up to 1.8 seconds, preferably up to 1.5 seconds, preferably up to 1.2 seconds, preferably up to 1 second, preferably up to 0.8 seconds, even more preferably up to 0.6 seconds and particularly preferably up to 0.5 seconds.

Different variants are possible for the power supply of the carbon crucible. In the first variant A, the mains current is only required for charging batteries or PowerCaps. This variant A corresponds to variants 3A, 3B and 4 in FIG. 1. Charging does not pose a significant challenge for the user's existing electricity system. Battery or PowerCap operation means that the dry aerosol can be produced in any room independently of the mains supply. The DC voltage of the battery or PowerCap is preferably transformed into a high-current AC voltage of approx. 10 volts.

Pulse width modulation/inverters are used to generate a sine wave/alternating voltage with the required high amount of energy (approx. 600 amperes) from the direct current sources, which enables the required fast risetime of the crucible.

In a second variant B (corresponds to variants 2A and 2B in FIG. 1), the mains current is supported/boosted by the batteries or PowerCaps during the risetime. This variant B corresponds to variants 2A and 2B in FIG. 1. This combination of mains current and energy storage therefore does not pose a significant challenge for the user's existing electricity system. With the battery or PowerCaps energy booster, the dry aerosol can be produced with almost any power connection. The DC voltage from the battery or PowerCaps/energy storage unit is converted into a 230 volt AC voltage using pulse width modulation/inverters. The AC voltage converted from the direct current sources/energy storage units is connected to the sine wave of the mains input in the device (socket current) almost in real time and boosts/increases the 230 volt mains current to well over 16 amps. This provides a transformer with the high energy briefly required for the required fast risetime of the crucible during fullburn. In a preferred embodiment of the invention, intelligent power current electronics analyze the sine wave of a 100 to 400 volt, preferably 110 to 260 volt, in particular 230 volt mains current and only switch on the power transformer for heating the carbon crucible when the switch-on load of the transformer reaches a defined, favorable section on the 50 Hz sine wave of the mains current. Preferably, a favorable section of the sine wave is a predetermined range of phase positions in which an inrush can be avoided when the transformer is switched on. The average person skilled in the art is able to identify such a favorable section and provide control means to switch on the transformer at the right time. This ensures that the transformer has the very high starting energy required to heat up the crucible (risetime) in the shortest possible time from a standard 230V socket without tripping the electrical fuse of the user's electricity system.

In preferred embodiments of the invention, electrical contacts for heating the carbon crucible to the firing temperature are supplied with an electrical current of up to 600 A at a voltage of 7-15, preferably about 10 volts. Preferably, this current is applied for the duration of the risetime and then reduced to a current of 200-400 A, preferably 300 A, while the temperature of the carbon crucible is maintained at the firing temperature. Preferably, the risetime is optimized using a sensing device and/or the control unit so that it is kept short. This can be achieved by intelligent control of a power board with a power supply from a standard socket.

In further preferred embodiments of the invention, the current frequency can be varied. Preferably, the current frequency can be set between 50-4000 Hz. This can improve the efficiency of the apparatus.

Optimized heating management of the carbon crucible can also help to reduce the variance in the dry aerosol quality. This minimizes fluctuations in the yield of the carbon crucible.

In preferred embodiments of the invention, the firing temperature is reached in less than 2 seconds, preferably up to 1.8 seconds, preferably up to 1.5 seconds, preferably up to 1.2 seconds, preferably up to 1 second, preferably up to 0.8 seconds, more preferably up to 0.6 seconds and particularly preferably up to 0.5 seconds. This increases the yield (amount of radioactivity in particle form in the dry aerosol) of the carbon crucible. As a result, less eluate and/or fewer breaths of the patient may be necessary for the detection of lung function.

In a further preferred embodiment of the invention, the apparatus comprises a detection device for monitoring the generation of dry aerosol in the combustion chamber. Preferably, a chamber activity measurement is performed to monitor the amount of radioactivity generated in the combustion chamber. This information can be forwarded to the control unit and/or optionally processed and shown on a display to assist the user. Preferably, the detection device comprises a gamma counter. This ensures that a sufficient amount of radioactive particles is present in the dry aerosol produced. The risk of user error, such as administering the dry aerosol too early or insufficiently filling the carbon crucible, is also minimized. Such an error can lead to the failure of a medical examination. In preferred embodiments of the invention, the apparatus comprises means for measuring the temperature of the carbon crucible, in particular during and after its heating. It may be particularly preferred that the temperature measurement is performed by measuring the light intensity of the glowing carbon crucible. This is particularly advantageous for precise measurement without contact with the crucible or the produced aerosol, so that neither the produced aerosol is contaminated nor the sensor damaged. Alternatively, a pyrometer can be used.

In a further preferred embodiment of the invention, the temperature of the produced radioactively labeled dry aerosol or inert gas is between 10-40° C., preferably between 15-30° C. At this temperature, the gas can be safely and comfortably inhaled by the patient without causing burns and is also safe for use with the various parts of the apparatus, e.g. disposable tubing and valves. It was also found that at these temperatures there was surprisingly little wear on the fixed parts of the apparatus such as the combustion chamber.

Preferably, the apparatus is shielded from the outside in order to protect the environment and all users and/or patients from radioactive radiation. The shielding can preferably be provided by means of lead and in particular surround the combustion chamber. In further embodiments, the shielding may preferably be provided by lead and/or tungsten and in particular surround the combustion chamber. In some embodiments, the shielding is provided by means of a lead housing.

In preferred embodiments of the invention, the apparatus comprises means for manual intervention in the control unit, preferably a touch screen. Preferably, the apparatus also comprises an output unit, such as a display, which indicates the steps of a procedure to be carried out. This allows the apparatus to be configured in a particularly user-friendly manner.

In a preferred embodiment of the invention, the eluate which is filled in the carbon crucible is dried. Preferably, this means that any water is removed from the eluate. Preferably, water vapor, ambient air and/or oxygen is removed by an evacuation process, for example by pumping water vapor, oxygen and/or air out of the combustion chamber.

In preferred embodiments of the invention, a combination of carbon, preferably from the carbon crucible, and an inert gas, preferably argon, is used to vaporize or atomize dried technetium eluate (sodium pertechnetate) in a chamber.

Preferably, the combustion chamber is filled with an inert gas before the carbon crucible is heated. This can be done by pumping inert gas into the chamber to displace the gas/air in the chamber, which can then escape through an exit channel. This step is preferably configured to remove any oxygen from the combustion chamber. Suitable inert gases are argon or nitrogen, wherein argon is particularly preferred.

It may be preferred that the apparatus comprises means for supplying the inert gas from a pressure vessel to the combustion chamber. Such means may include a duct, a controllable valve and/or a controllable pump. Preferably, the pressure vessel is located on a movable trolley which is part of the apparatus or can be used with the apparatus.

Preferably, the apparatus comprises means for generating compressed air and/or a vacuum. This may comprise, for example, a pump and/or a pressure vessel.

Preferably, the apparatus comprises a battery or rechargeable power source which can be used in an optional step of administering the dry aerosol to a patient. This is particularly advantageous for ensuring that the apparatus is freely movable during this phase.

In a further preferred embodiment, the apparatus comprises a mouthpiece, a patient tube, a patient outlet valve for selectively discharging gas or dry aerosol from the combustion chamber into the patient tube and/or a ventilation valve for selectively admitting room air into the combustion chamber to create a flow through the combustion chamber into the patient tube. A “patient tube” in the context of the invention is preferably a tube in which the aerosol is directed from the combustion chamber for administration to the patient. Preferably, the mouthpiece and the patient tube are configured as disposable components.

In a further preferred embodiment, the apparatus comprises an outlet valve for evacuating the combustion chamber, in particular for removing water vapor, residual oxygen and purge gas from the combustion chamber. In a further preferred embodiment, the apparatus comprises an inlet valve for filling the combustion chamber with an inert gas and/or with compressed air.

In a preferred embodiment of the apparatus, the combustion chamber is equipped with a closable opening for manual insertion of tools, e.g. a syringe, in particular to fill the carbon crucible.

In a further preferred embodiment of the invention, the apparatus comprises means for remote access by a technician, in particular to the control unit and any input or feedback units such as touch screen, display, keypad, alarm, light, etc. Preferably, the apparatus also comprises means for connection to a local or wireless network, in particular a Bluetooth, radio, satellite or WLAN connection.

In a further preferred embodiment of the invention, the device comprises a data memory. Preferably, the control unit is configured to store data from one or more detection devices in the data memory. In this way, the course of a procedure can be automatically logged via the device. Values such as, for example, the amount of activity filled in, temperature attainment and progression, number of breaths, volume of breaths, measured activity in the chamber and the increase in activity in the lungs can be recorded and logged for each patient by the device. These values should serve as a log of the examination.

In a second aspect, the invention relates to a method for producing radioactively labeled dry aerosol comprising
a. providing an apparatus for producing radioactively labeled dry aerosol according to the first aspect of the invention,
b. carrying out a breathing test (pre-breathing) on the patient, wherein the patient's respiratory volume is measured to assess their fitness for examination,
c. camera-monitored filling of a carbon crucible inserted into the combustion chamber of the apparatus with a radioactive nuclide,
d. optional pressure test to check the seal of the combustion chamber,
e. optional vaporization of a carrier liquid of the radioactive nuclide,
f. optional drying of the radioactive nuclide,
g. evacuation of the air from the combustion chamber to create a vacuum
h. filling of the combustion chamber with argon gas, with simultaneous monitoring of any residual oxygen present, to generate an inert argon, nitrogen or shielding-gas atmosphere in the combustion chamber,
i. preheating of the carbon crucible to preferably 500° C.,
j. Heating the filled carbon crucible to 2,500-3,000° C., preferably 2,750° C. to produce a radioactively labeled dry aerosol or nanoparticle composite, wherein the electricity required during heating is provided, either
I. by the at least one battery and/or PowerCap/buffer capacitor and/or high-performance capacitor contained therein, or
II. by mains current, which is supported and optionally amplified by the at least one battery and/or PowerCap/buffer capacitor and/or high-performance capacitor, or
III. by mains current, wherein the intelligent power electronics unit analyzes the sine wave (preferably 50 Hz) of the alternating voltage of the mains current (preferably 230 volts) and only switches the transformer on when the inrush load of the transformer reaches a predefined, favorable point in time on the sine wave of the mains current, so that the filled carbon crucible is heated to 2,500-3,000° C., preferably 2,750° C., preferably within up to 2 seconds, preferably up to 1.8 seconds, preferably up to 1.5 seconds, preferably up to 1.2 seconds, preferably up to 1 second, preferably up to 0.8 seconds, even more preferably up to 0.6 seconds and particularly preferably up to 0.5 seconds,
k. optional monitoring of the change in radioactivity in the combustion chamber
l. lung activity monitoring during inhalation by a patient of the produced radioactively labeled dry aerosol or nanoparticle composite, wherein the amount of radioactivity inhaled by the patient is monitored by a radioactivity sensor, preferably localized on the patient,
m. purging of the combustion chamber with compressed air to remove the radioactive particles, wherein the radioactive particles are removed by a filter from the outflowing air, argon, nitrogen or shielding gas mixture, and optional monitoring of the change in radioactivity in the combustion chamber,
n. optional automatic logging of one or more process parameters.

In a preferred embodiment of the process, a new carbon crucible is inserted into the combustion chamber for each pass of the process. The insertion of the carbon crucible into the combustion chamber is preferably carried out manually. It may also be advantageous that the apparatus comprises means for automatically positioning the carbon crucible in the combustion chamber and automatically engaging the carbon crucible with electrical contacts.

In a preferred embodiment of the method, the radioactive nuclide is selected from technetium (Tc-99m) or gallium 68.

In a further preferred embodiment of the method, the radioactive nuclide is present in the form of a liquid eluate. Preferably, the eluate is introduced manually into the cavity of the carbon crucible. Preferably, this step is monitored by a camera. Preferably, a live monitored image from the camera is shown on a display. This not only enables a better view and the possibility of enlarging the monitored image for better precision, but also increases the safety of the user, as no body parts, but only tools, need to enter the combustion chamber. As the combustion chamber can be enclosed in thick lead shielding, visibility is greatly improved due to the camera, preventing spillage, waste and damage to the apparatus.

Preferably, the user can look at a display that shows him/her via camera the live image of his/her activity in the device drawer in real time on the monitor screen. A variable zoom function allows the user to visually enlarge their actions if required. Camera monitoring eliminates the need to look through the thick shielding lead glass. The necessary radiation shielding to the user can now be easily realized by an appropriate lead plate shielding, which also allows the use of higher energy nuclides such as gallium.

In a further preferred embodiment of the method, a pressure test is carried out to check the seal of the combustion chamber. Preferably, the combustion chamber is filled with air or an inert gas at an overpressure of 75-500 mbar, preferably 100-300 mbar, in particular approx. 120 mbar. The combustion chamber preferably comprises closable valves, which are closed after filling with air or an inert gas. The course of the pressure drop is monitored, e.g. by means of a pressure gauge. The data from the pressure gauge are preferably supplied to the control unit. If the rate of pressure drop exceeds a predetermined value, the control unit preferably triggers a fault signal. This can take the form of an alarm, a light signal, a message on a display or similar.

In another preferred embodiment of the method, the water is vaporized from the carbon crucible during a vaporization phase. The water may make up most of the eluate and has approximately the same volume as the eluate. For example, if 0.3 ml of eluate is placed in a carbon crucible with a capacity of 0.3 ml, the volume of the vaporized water is preferably close enough to 0.3 ml that it can be approximated as 0.3 ml. Preferably, the carbon crucible is heated to 70-95° C., preferably about 85° C., during the vaporization phase. Preferably, this phase lasts 3-6 minutes, particularly preferably approx. 4 minutes. During this time, the resulting “water vapor” is transported out of the chamber by the flowing compressed air and is evacuated from the combustion chamber through an open outlet valve, which is preferably arranged downstream of a device filter in the direction of flow.

In a further preferred embodiment of the method, during a safety drying phase (also “TTM” or crucible temperature management in the context of the invention), a diffused residual water component is driven out of any pores of the carbon crucible by heating the carbon crucible to 150-200° C., in particular approx. 180° C. Preferably, this process takes 0.5-2 minutes, in particular approx. 1 minute. During this phase, the resulting “water vapor” (steam) is conveyed out of the combustion chamber by compressed air flowing through it and is evacuated through the open outlet valve behind the device filter.

In a further preferred embodiment, the apparatus comprises a vacuum pump and/or a compressor for conserving inert gas resources. Preferably, the air is pumped out of the combustion chamber in this method. This can create a vacuum in the combustion chamber. The subsequent build-up of a protective atmosphere in the combustion chamber is thus shortened and facilitated. The amount of argon or inert gas required can thus be greatly reduced, thereby lowering the cost of consumables.

Preferably, a compressor is integrated in the combustion chamber. The compressor can preferably generate compressed air. Processes in the combustion chamber for which no inert shielding gas is required are now performed with simple filtered compressed air. The compressed air is generated by the apparatus itself. The amount of inert gas required, in particular argon, can be greatly reduced, thus cutting the cost of consumables.

In a further preferred embodiment of the method, the combustion chamber is filled with an inert gas, preferably argon. After this step, preferably no oxygen remains in the combustion chamber. Preferably, an excess of inert gas is supplied to the combustion chamber, with some of the inert gas leaving the combustion chamber via the open outlet valve. This process allows the oxygen-containing air in the combustion chamber to be displaced by the inert gas so that no oxygen remains.

In a further preferred embodiment of the method, an oxygen content is monitored in the combustion chamber and/or in an outlet duct from the combustion chamber. Preferably, measured data of the oxygen content is supplied to the control unit. In one example, an oxygen measuring probe is used at a chamber outlet. Preferably, the control unit outputs a signal if the determined oxygen content exceeds or falls below a predefined limit value. For example, a message is shown on a display. The carbon crucible can only be heated to the firing temperature when the oxygen content falls below the specified limit value

In a further preferred embodiment of the method, any valves of the combustion chamber are closed before the carbon crucible is heated to the firing temperature.

In a further preferred embodiment, the method comprises the step of preheating the carbon crucible. Preferably, the carbon crucible is first heated to a temperature between 250-800° C., preferably 350-650° C., particularly preferably approx. 500° C. This reduces the risetime and improves the efficiency of the method.

In a further preferred embodiment of the method, the carbon crucible is heated to the firing temperature by abrupt heating. Preferably, the firing temperature is 2500-3000° C., in particular approx. 2750° C. Preferably, the firing temperature is reached within a risetime of up to 2 seconds, preferably up to 1.8 seconds, preferably up to 1.5 seconds, preferably up to 1.2 seconds, preferably up to 1 second, preferably up to 0.8 seconds, more preferably up to 0.6 seconds and particularly preferably up to 0.5 seconds.

In a further preferred embodiment of the method, the risetime is reduced. Preferably, a risetime of up to 2 seconds, preferably up to 1.8 seconds, preferably up to 1.5 seconds, preferably up to 1.2 seconds, preferably up to 1 second, preferably up to 0.8 seconds, even more preferably up to 0.6 seconds and particularly preferably up to 0.5 seconds is implemented using intelligent power electronics with a mains and/or energy storage operation. Preferably, heating can be carried out with the aid of an accumulator, a battery, a PowerCap, a buffer capacitor and/or a high-performance capacitor.

In a further preferred embodiment of the method, during the heating of the filled carbon crucible under point j. the direct current of the at least one battery and/or PowerCap/buffer capacitor and/or high-performance capacitor is converted into an alternating voltage (preferably 230 volts) by means of pulse width modulation or an inverter, such that the voltage converted from the at least one battery and/or PowerCap/buffer capacitor and/or high-performance capacitor is connected to the sine wave of the alternating voltage (preferably 230 volts) of the mains input of the apparatus and thus amplifies the mains current to preferably ≥16 amperes.

In a further preferred embodiment of the method, a resulting change in radioactivity in the combustion chamber is monitored by one or more detection devices. Preferably, the recorded data is forwarded to the control unit. Preferably, the control unit outputs signals to indicate reaching a certain phase in the process of producing the dry aerosol. It may also be preferred that the control unit instructs a display to show a message to the user based on the data collected. Once it is determined that radioactively labeled dry aerosol is substantially being produced (e.g., when the level of radioactivity exceeds a predetermined threshold), the dry aerosol is preferably administered to a lung within 15 minutes, more preferably within 10 minutes.

In a further preferred embodiment of the method during the breathing test under point b. the patient breathes through a set of tubes, preferably connected to the apparatus, such that when breathing in, filtered room air flows through a flow meter, preferably integrated in the apparatus, which measures the inhaled volume per breath.

In a further preferred embodiment of the method, the combustion chamber is ventilated so that a patient can inhale the radioactively labeled dry aerosol in one to five breaths. Ventilation is preferably provided by a ventilation valve, which selectively allows air to enter the combustion chamber. The radioactively labeled dry aerosol is preferably fed from the combustion chamber to the patient through an open patient outlet valve and a “patient tube”. With each breath, room air flows into the chamber through the open ventilation valve. If there is sufficient activity in the lungs, the patient outlet valve is closed and administration is completed.

In another preferred embodiment of the method, the patient's actual breathing volume is measured to assess the patient's suitability for examination. The data from this “pre-breathing” measurement can be fed to the control unit and shown on the display.

It is a well-known problem worldwide that when handling a radioactively labeled dry aerosol, the user often does not know in advance whether the patient will be able to take the necessary 1 to 5 deep breaths with sufficient inhalation volume. Experienced users can assess this better than inexperienced users. It is often the case that the procedure has to be aborted. This is where our new pre-breathing monitoring comes in. In preparation, the patient breathes through the connected tube set. When breathing in, filtered room air (without activity) flows through a bypass past the combustion chamber into the patient tube. An integrated flow meter measures the inhaled volume per breath and supports the user in deciding whether the patient's breathing volume is sufficient for a successful examination. This procedure saves the user time and is safer for the patient. The patient does not run the risk of being exposed to radioactivity for an aborted, inconclusive examination. For the user, in particular, time is saved for incorrect examinations.

If the patient is unable to breathe deeply enough even after being prompted to do so, the user can immediately use a supportive ventilation bag on the apparatus.

In the context of the present invention, the ventilation bag is preferably a bag that can be squeezed to force air through the combustion chamber and the patient tube and deliver it to the patient. This is particularly useful when the patient is unable to produce sufficient suction to generate this airflow.

When the ventilation bag is compressed, an outlet valve in the bag opens automatically. The air is forced out of the bag into the combustion chamber. Preferably, the air flow automatically opens a valve that allows the air to flow through the patient tube. The radioactively labeled aerosol is thus delivered to the patient in sufficient quantity before exhalation can take place.

The ventilation bag is preferably released when the patient exhales. The ventilation bag preferably comprises means to reinflate itself, e.g. a special inflation valve. This process can be repeated as often as is deemed safe and necessary.

In another preferred embodiment of the method, the inhaled amount of dry aerosol in a lung is monitored. This can be done using a detection device or sensor to detect the decay of positrons over time. The results can be stored in a memory and/or shown on a display.

In a further preferred embodiment of the method, the dry aerosol is purged from the combustion chamber after the carbon crucible has been used once. Preferably, the purging is carried out with compressed air or inert gas from a pressure vessel or with the aid of a compressor. The compressed air or inert gas can displace the dry aerosol, which is preferably disposed of safely. This is preferably done using a lead-shielded device filter. Preferably, the method may also include a step of mechanically destroying the carbon crucible and/or radioactively protected storage and disposal of disposable components such as a tube for delivering the dry aerosol to a patient.

In a further preferred embodiment of the method, a detection device (“activity sensor”) for detecting the radioactive activity, in particular for detecting the decay of positrons over time, is mounted in a vest. The vest can be used with the apparatus according to the invention. The vest can be used to record the increase in activity in the patient's lungs when the radioactive dry aerosol is administered. During the examination, preferably for performing a ventilation scintigraphy, the patient wears this vest, which is preferably connected to the apparatus.

In the context of the present invention, the vest is preferably characterized in that it comprises a plurality of spatially distributed sensors and means for attachment to the front, left side, right side and/or back of the thorax region of the patient. The vest may, for example, be designed as a bib with a neck strap, an apron, an enlarged belt, a shirt or a garment with an opening for the head between a front and a back part. Preferably, the vest comprises adjustable fastening means to allow a close fit for patients with different body shapes and sizes. Preferably, the vest comprises an opening for the head located between a front and a back portion and Velcro fasteners for securing the front and back portions at the sides and/or under the armpits of the patient to ensure a good fit.

Preferably, a target value of the desired amount of activity in the patient's lungs is specified by the user. This value is individual and also depends, for example, on the gamma camera and/or PET camera that the user can subsequently use for imaging.

The user thus preferably defines the “target criterion” at the time of administration to ensure that only the desired amount of activity reaches the lungs. The valves and/or pumps of the apparatus can be controlled by the control unit based on this predetermined data to ensure that just enough dry aerosol arrives in the patient's lungs to perform the desired analysis/examination, but no more than necessary to avoid unnecessarily exposing the patient to radioactivity. It is also advantageous for an optional second part of the examination, the perfusion scintigraphy for visualizing the blood flow in the lungs, that not too much radioactively labeled dry aerosol has reached the lungs during the ventilation scintigraphy, as otherwise the permissible total radiation dose could be exceeded and the second directly subsequent part of the examination (perfusion scintigraphy) may not be able to be performed at this time and would have to be postponed.

If the desired activity setpoint is measured by the sensor on the patient's chest, the control unit sends a signal. The patient valve is closed. No further activity can be inhaled. The administration of the radioactive dry aerosol is terminated.

In a third aspect, the invention relates to a use of the apparatus according to the invention for imaging the ventilated regions of a lung, preferably using a gamma camera and/or PET camera.

In a preferred embodiment of the invention, the imaging of the ventilated regions of a lung is performed by pulmonary ventilation scintigraphy. It may be preferred that the image-based representation is a tomographic representation.

In certain embodiments, the apparatus according to the invention can also be used to detect blockages and/or leaks in a non-living airflow system, e.g. in a microflow reactor. Also in such an application, the invention can be used to image a system ventilation, preferably using a gamma camera and/or PET camera.

In a fourth aspect, the invention relates to an apparatus comprising a combustion chamber, a current pulse generator, a control unit, a probe for measuring the oxygen content in the combustion chamber, a vacuum pump, a compressed air compressor, a camera and a power supply unit as described above. In particular in this aspect, the carbon crucible is not part of the apparatus but is a consumable component that can be used with the apparatus. Preferably, the apparatus is configured to accommodate different shapes and sizes of carbon crucibles.

In a fifth aspect, the invention relates to a system for producing a radioactively labeled dry aerosol, wherein the system comprises an apparatus according to the fourth aspect in combination with a carbon crucible, a compressed air container, an inert gas pressure vessel, a disposable patient tube and/or an external ventilation bag.

The average person skilled in the art will recognize that technical features, definitions and advantages of preferred embodiments of the method according to the invention also apply to the apparatus and system according to the invention, and vice versa.

Terms such as substantially, approximately, about, approx., etc. preferably describe a tolerance range of less than ±20%, preferably less than ±10%, particularly preferably less than ±5% and in particular less than ±1% and include the exact value.

### EXAMPLES

### Example 1

Overview of the measurement results of the preliminary tests carried out for each test variant as shown in FIG. 1

Description of the test setup and procedure for the tests carried out for the purpose of generating the dry aerosol

### Components:

Test combustion chamber with dome
Test power sources
Test power electronics
Test control electronics
Carbon crucible
Carbon contacts
Salt solution
Inert gas/argon
Oxygen measuring unit
Pressure pump
Vacuum pump
Test filter
Radioactive nuclide e.g. technetium eluate

### Test Procedure Part 1

The test was carried out with saline solution without a radioactive nuclide. In addition, the electrical equipment used, consisting of the energy source and electronics, was tested for function and heating speed of the crucible. These tests were carried out in the electronics laboratory and serve to verify the electro-thermal function of the respective variant.

### Test Procedure Part 2

The test was carried out with salt solution and with a radioactive nuclide (technetium). In addition, the electrical equipment used, consisting of energy source and electronics, was tested for function and heating speed of the crucible, as well as the radioactivity emitted as radioactively labeled carbon particles. (The tests with radioactivity could only be carried out in a nuclear facility, e.g. nuclear medicine department).

### Preparation

Inserting the carbon crucible into the combustion chamber Manual filling of the crucible with active eluate (max. 0.3 ml) New: Camera-based manual filling of the crucible Innovative user protection/radiation protection

### Leak Test

Pressure test to check the seal of the chamber. The chamber is filled with an overpressure of 120 mbar. All valves are closed and the progression of the pressure drop is evaluated. If the pressure drop is too rapid, the apparatus issues an error message regarding the chamber seal.

### Vaporization

The water content (approx. 0.3 ml) is removed from the radioactive eluate by heating the crucible to approx. 85° C. The process takes approx. 4 minutes. During this time, the resulting “water vapor” is transported out of the chamber by the flowing compressed air and is evacuated through the open device outlet valve downstream of the device filter.

### Drying

New: Safety drying (TTM crucible temperature management) The diffused residual water is expelled from the pores by heating the crucible to approx. 180° C. The process takes approx. 1 minute. During this time, the resulting “water vapor” is transported out of the chamber by the flowing compressed air and escapes through the open device outlet valve downstream of the device filter.

### Evacuation (New Process Step)

New: Integration of a vacuum pump and compressor to conserve argon gas resources.
The air is pumped out of the chamber. A vacuum is created in the chamber.

### Inert Atmosphere

The chamber is filled with argon; no oxygen may remain in the chamber.
Excess argon is evacuated through the open device outlet valve downstream of the device filter.
New: The argon flowing out of the open device outlet valve is checked for residual oxygen using an oxygen measuring unit.

### Closing the Valves

The chamber is filled with an inert shielding gas atmosphere without pressure.

### Preheating (New Process Step)

New: Preheating the crucible to e.g. 500° C. (to accelerate the subsequent risetime)

### Firing Process

The Ventigas dry aerosol is generated by abruptly heating the crucible to approx. 2750° C. The heating time/risetime should only take 0.5 to 0.8 seconds!
New: Risetime acceleration (reduction of the heating time of the crucible)
New: Intelligent power electronics with mains and/or energy storage operation
New: Chamber activity monitoring
For monitoring the resulting radioactivity change in the apparatus chamber in order to help the user (assistance system to help the user)
Note: The time window for administration to the patient from the firing process is max. 10 minutes. The concentration of particles in the gas mixture decreases over time! In the tests, the samples are taken one minute after the firing process.
   Administration/Suction into the Test Filter During Tests
Suction of the active Ventigas dry aerosol through the open patient outlet valve and a “patient tube” from the ventilated chamber into a test filter. During suction, room air flows into the chamber through the open ventilation valve. After air exchange of three times the chamber volume, the free particles with the activity are in the test filter. The patient outlet valve is closed.

### Activity Evaluation

Evaluation of the amount of radioactivity of the extracted particles in the test filter compared to the standard application.

### Flushing the Chamber

Residual active Ventigas is removed from the generator chamber by flushing with compressed air from the new device compressor. Any remaining suspended particles with activity are flushed into the device filter and safely filtered out. Argon and air are evacuated via the open device outlet valve.
New: Integration of a compressor to conserve argon gas resources.
New: Chamber activity monitoring
For monitoring the resulting change in radioactivity in the device chamber.

Examination of the different variants of power sources and electronic setups for producing the radioactively labeled carbon particles/radioactive dry aerosol (variants according to FIG. 1)

The tests of the different variants (FIG. 1) of power sources and electronic setups for producing the radioactively labeled carbon particles/the radioactive dry aerosol were carried out in nuclear medicine facilities. Only the technical nuclear medicine equipment and radioactive nuclides were used in these tests. Patients did not come into contact with the tests.

### Nuclear Medicine Facilities:

Harz PET Center & Nuclear Medicine
Dr. med. Frank Straube
Kösliner Strasse 12
38642 Goslar
Bethesda Evangelical Hospital in Duisburg
Clinic for Nuclear Medicine
Heerstrasse 219
47053 Duisburg

TABLE 1
Overview of the measurement results of the preliminary
tests carried out for each variant according to FIG. 1

| | Measurement 1 | Measurement 2 | Measurement 3 | Mean values | |
|---|---|---|---|---|---|
| Standard | 2720 | 2700 | 2660 | 2693.3 | °C |
| application | 36.9 | 42.7 | 51.1 | 43.6 | cnts/s/MBq |
| for comparison | | | | | |
| Variant 1 | 2730 | 2710 | 2670 | 2703.3 | °C |
| | 41.0 | 45.5 | 45.6 | 44.0 | cnts/s/MBq |
| Variant 2A | 2700 | 2690 | 2690 | 2693.3 | °C |
| | 40.2 | 46.7 | 37.8 | 41.5 | cnts/s/MBq |
| Variant 2B | 2724 | 2740 | 2696 | 2720.0 | °C |
| | 43.8 | 48.9 | 47.0 | 46.6 | cnts/s/MBq |
| Variant 3A | 2750 | 2690 | 2690 | 2710.0 | °C |
| | 44.1 | 43.7 | 40.4 | 42.7 | cnts/s/MBq |
| Variant 3B | 2710 | 2700 | 2680 | 2696.7 | °C |
| | 42.0 | 44.1 | 39.4 | 41.8 | cnts/s/MBq |
| Variant 4 | 2650 | 2600 | 2627 | 2625.7 | °C |
| | 36.1 | 27.2 | 45.1 | 36.1 | cnts/s/MBq |

### Example 2

Comparative measurements for demonstrating the effectiveness of the developed “intelligent power electronics (IPE)” in the dry aerosol generator

Tests with a functional model have shown that when the transformer is switched on in an “uncontrolled, random” manner in the firing process, the inrush current can be a multiple of the rated current.

Further tests have shown that “controlled” switching on and off by an intelligent control system has reduced the inrush current to such an extent that it no longer deviates significantly from the rated current (see Table 2 and FIG. 2).

The series of measurements show the large difference in the level and divergence of the inrush current when the transformer is switched on for the firing process. One series of measurements was carried out without and one with the use of the “intelligent power electronics (IPE)”.

TABLE 2

| | without IPE | with IPE |
|---|---|---|
| Measurement | (in | (in |
| no. | amperes) | amperes) |
| 1 | 38.2 | 14.2 |
| 2 | 32.8 | 13.9 |
| 3 | 14.3 | 14.1 |
| 4 | 48.3 | 14.3 |
| 5 | 24.6 | 13.9 |
| 6 | 42.7 | 14.2 |
| 7 | 18.6 | 14 |
| 8 | 35.6 | 14.6 |
| 9 | 26.9 | 14.1 |
| 10 | 39.7 | 15.1 |
| 11 | 34.4 | 14.6 |

### Example 3

Chamber activity measurement for monitoring the amount of radioactivity produced in the combustion chamber

Tests with a combustion chamber, as preferably used in the preferred embodiment of the invention, have shown that the increase in activity in the chamber can be determined and evaluated by means of a gamma counter. This makes it possible to record the increase in activity and thus to assess the activity yield of the dry aerosol produced by using appropriate evaluation software, which allows conclusions to be drawn about the quality of the dry aerosol (see FIG. 3).

### Example 4

Monitoring the residual oxygen content in the combustion chamber

Furthermore, a process measurement was carried out to monitor the residual

oxygen content during the build-up of the inert gas/shielding gas atmosphere in the combustion chamber of the dry aerosol generator/Ventigas generator, i.e. during the process of filling the combustion chamber with argon gas.

The aim is to generate an inert argon, nitrogen or shielding gas atmosphere in the combustion chamber in order to be able to carry out the subsequent heating process of the carbon crucible without the presence of oxygen. In order to be able to make reliable statements about the quality of the shielding gas atmosphere and to ensure a stable, reproducible process, continuous quality control of the shielding gas atmosphere is necessary.

A suitable oxygen measuring cell was integrated into the test setup of the device chamber in a test to demonstrate its feasibility. With this measuring cell, the oxygen content in the combustion chamber could be quantified and evaluated as required in the test when the shielding gas atmosphere was built up (see Table 3 and FIGS. 4 and 5).

The series of measurements shows the change in the residual oxygen content in the combustion chamber during the build-up of the shielding gas atmosphere. The residual oxygen content is given in ppm (parts per million). Over time, the residual oxygen content continues to decrease and the concentration of the shielding gas atmosphere increases. With the new measuring apparatus, the exact point in time can be determined at which a specified extremely low limit value is reached and the next process step can start. This is of great importance for the continuous and reproducible high quality of the dry aerosol.

FIG. 4 shows the series of measurements as a graphical measurement curve in the form of a diagram over the entire course of the measurement of the residual oxygen content when the inert gas-shielding gas atmosphere is created in the combustion chamber of the dry aerosol generator. The “resolution” of the diagram in the y-direction (O2 in ppm) is shown here roughly in steps of 10,000.

FIG. 5 shows the series of measurements as a graphical measurement curve in the form of a diagram of the end of the measurement curve of the residual oxygen content during the build-up of the inert gas-shielding gas atmosphere in the combustion chamber of the dry aerosol generator. The “zoomed resolution” of the diagram in the y-direction (O2 in ppm) is displayed in finer increments of 100.

TABLE 3

| Measuring | Residual | Inert | Inert |
|---|---|---|---|
| time | oxygen | gas | gas |
| Time | Oxygen O2 | flow | flow |
| in sec. | in ppm | in sec. | in min. |
| 0 | 209400 | 0 | 0.00 |
| 2 | 209400 | 0 | 0.00 |
| 4 | 209400 | 0 | 0.00 |
| 6 | 209400 | 0 | 0.00 |
| 8 | 209400 | 0 | 0.00 |
| 10 | 209400 | 0 | 0.00 |
| 12 | 209400 | 0 | 0.00 |
| 14 | 209400 | 0 | 0.00 |
| 16 | 209400 | 0 | 0.00 |
| 18 | 209400 | 0 | 0.00 |
| 20 | 209400 | 0 | 0.00 |
| 22 | 209400 | 0 | 0.00 |
| 24 | 209400 | 0 | 0.00 |
| 26 | 209400 | 0 | 0.00 |
| 28 | 209400 | 0 | 0.00 |
| 30 | 209400 | 0 | 0.00 |
| 32 | 209400 | 0 | 0.00 |
| 34 | 209400 | 0 | 0.00 |
| 36 | 209400 | 0 | 0.00 |
| 38 | 209400 | 0 | 0.00 |
| 40 | 209400 | 0 | 0.00 |
| 42 | 209400 | 0 | 0.00 |
| 44 | 209400 | 0 | 0.00 |
| 46 | 209400 | 0 | 0.00 |
| 48 | 209400 | 0 | 0.00 |
| 50 | 209400 | 0 | 0.00 |
| 52 | 209400 | 0 | 0.00 |
| 54 | 209400 | 0 | 0.00 |
| 56 | 209400 | 0 | 0.00 |
| 58 | 209400 | 0 | 0.00 |
| 60 | 209400 | 0 | 0.00 |
| 62 | 209400 | 0 | 0.00 |
| 64 | 209400 | 0 | 0.00 |
| 66 | 209400 | 2 | 0.03 |
| 68 | 209400 | 4 | 0.07 |
| 70 | 191503 | 6 | 0.10 |
| 72 | 179621 | 8 | 0.13 |
| 74 | 169930 | 10 | 0.17 |
| 76 | 159990 | 12 | 0.20 |
| 78 | 149027 | 14 | 0.23 |
| 80 | 139231 | 16 | 0.27 |
| 82 | 131049 | 18 | 0.30 |
| 84 | 121411 | 20 | 0.33 |
| 86 | 113444 | 22 | 0.37 |
| 88 | 106578 | 24 | 0.40 |
| 90 | 99074 | 26 | 0.43 |
| 92 | 93774 | 28 | 0.47 |
| 94 | 89138 | 30 | 0.50 |
| 96 | 83868 | 32 | 0.53 |
| 98 | 78404 | 34 | 0.57 |
| 100 | 75384 | 36 | 0.60 |
| 102 | 72226 | 38 | 0.63 |
| 104 | 66836 | 40 | 0.67 |
| 106 | 62990 | 42 | 0.70 |
| 108 | 60728 | 44 | 0.73 |
| 110 | 57699 | 46 | 0.77 |
| 112 | 54415 | 48 | 0.80 |
| 114 | 51356 | 50 | 0.83 |
| 116 | 48785 | 52 | 0.87 |
| 118 | 46623 | 54 | 0.90 |
| 120 | 44591 | 56 | 0.93 |
| 122 | 42205 | 58 | 0.97 |
| 124 | 40184 | 60 | 1.00 |
| 126 | 38691 | 62 | 1.03 |
| 128 | 36995 | 64 | 1.07 |
| 130 | 34916 | 66 | 1.10 |
| 132 | 32888 | 68 | 1.13 |
| 134 | 31339 | 70 | 1.17 |
| 136 | 29436 | 72 | 1.20 |
| 138 | 27786 | 74 | 1.23 |
| 140 | 26446 | 76 | 1.27 |
| 142 | 25425 | 78 | 1.30 |
| 144 | 24274 | 80 | 1.33 |
| 146 | 23713 | 82 | 1.37 |
| 148 | 22487 | 84 | 1.40 |
| 150 | 21028 | 86 | 1.43 |
| 152 | 19540 | 88 | 1.47 |
| 154 | 18531 | 90 | 1.50 |
| 156 | 18330 | 92 | 1.53 |
| 158 | 17988 | 94 | 1.57 |
| 160 | 17124 | 96 | 1.60 |
| 162 | 15875 | 98 | 1.63 |
| 164 | 14944 | 100 | 1.67 |
| 166 | 14092 | 102 | 1.70 |
| 168 | 13539 | 104 | 1.73 |
| 170 | 13090 | 106 | 1.77 |
| 172 | 12944 | 108 | 1.80 |
| 174 | 12269 | 110 | 1.83 |
| 176 | 11595 | 112 | 1.87 |
| 178 | 11391 | 114 | 1.90 |
| 180 | 10898 | 116 | 1.93 |
| 182 | 10439 | 118 | 1.97 |
| 184 | 10014 | 120 | 2.00 |
| 186 | 9555 | 122 | 2.03 |
| 188 | 9227 | 124 | 2.07 |
| 190 | 8915 | 126 | 2.10 |
| 192 | 8341 | 128 | 2.13 |
| 194 | 7925 | 130 | 2.17 |
| 196 | 7646 | 132 | 2.20 |
| 198 | 7304 | 134 | 2.23 |
| 200 | 6949 | 136 | 2.27 |
| 202 | 6574 | 138 | 2.30 |
| 204 | 6294 | 140 | 2.33 |
| 206 | 5852 | 142 | 2.37 |
| 208 | 5755 | 144 | 2.40 |
| 210 | 5505 | 146 | 2.43 |
| 212 | 5221 | 148 | 2.47 |
| 214 | 5155 | 150 | 2.50 |
| 216 | 4828 | 152 | 2.53 |
| 218 | 4668 | 154 | 2.57 |
| 220 | 4511 | 156 | 2.60 |
| 222 | 4427 | 158 | 2.63 |
| 224 | 4277 | 160 | 2.67 |
| 226 | 4161 | 162 | 2.70 |
| 228 | 4032 | 164 | 2.73 |
| 230 | 3712 | 166 | 2.77 |
| 232 | 3400 | 168 | 2.80 |
| 234 | 3261 | 170 | 2.83 |
| 236 | 3139 | 172 | 2.87 |
| 238 | 3010 | 174 | 2.90 |
| 240 | 2905 | 176 | 2.93 |
| 242 | 2809 | 178 | 2.97 |
| 244 | 2727 | 180 | 3.00 |
| 246 | 2577 | 182 | 3.03 |
| 248 | 2449 | 184 | 3.07 |
| 250 | 2391 | 186 | 3.10 |
| 252 | 2252 | 188 | 3.13 |
| 254 | 2098 | 190 | 3.17 |
| 256 | 2053 | 192 | 3.20 |
| 258 | 1983 | 194 | 3.23 |
| 260 | 1882 | 196 | 3.27 |
| 262 | 1843 | 198 | 3.30 |
| 264 | 1725 | 200 | 3.33 |
| 266 | 1558 | 202 | 3.37 |
| 268 | 1521 | 204 | 3.40 |
| 270 | 1453 | 206 | 3.43 |
| 272 | 1360 | 208 | 3.47 |
| 274 | 1317 | 210 | 3.50 |
| 276 | 1259 | 212 | 3.53 |
| 278 | 1181 | 214 | 3.57 |
| 280 | 1130 | 216 | 3.60 |
| 282 | 1088 | 218 | 3.63 |
| 284 | 1043 | 220 | 3.67 |
| 286 | 982 | 222 | 3.70 |
| 288 | 939 | 224 | 3.73 |
| 290 | 891 | 226 | 3.77 |
| 292 | 851 | 228 | 3.80 |
| 294 | 819 | 230 | 3.83 |
| 296 | 774 | 232 | 3.87 |
| 298 | 698 | 234 | 3.90 |
| 300 | 663 | 236 | 3.93 |
| 302 | 640 | 238 | 3.97 |
| 304 | 610 | 240 | 4.00 |
| 306 | 576 | 242 | 4.03 |
| 308 | 556 | 244 | 4.07 |
| 310 | 538 | 246 | 4.10 |
| 312 | 521 | 248 | 4.13 |
| 314 | 492 | 250 | 4.17 |
| 316 | 456 | 252 | 4.20 |
| 318 | 434 | 254 | 4.23 |
| 320 | 408 | 256 | 4.27 |
| 322 | 377 | 258 | 4.30 |
| 324 | 350 | 260 | 4.33 |
| 326 | 329 | 262 | 4.37 |
| 328 | 304 | 264 | 4.40 |
| 330 | 291 | 266 | 4.43 |
| 332 | 274 | 268 | 4.47 |
| 334 | 251 | 270 | 4.50 |
| 336 | 246 | 272 | 4.53 |
| 338 | 230 | 274 | 4.57 |
| 340 | 200 | 276 | 4.60 |
| 342 | 185 | 278 | 4.63 |
| 344 | 168 | 280 | 4.67 |
| 346 | 149 | 282 | 4.70 |
| 348 | 139 | 284 | 4.73 |
| 350 | 123 | 286 | 4.77 |
| 352 | 112 | 288 | 4.80 |
| 354 | 106 | 290 | 4.83 |
| 356 | 92 | 292 | 4.87 |
| 358 | 83 | 294 | 4.90 |
| 360 | 71 | 296 | 4.93 |
| 362 | 57 | 298 | 4.97 |
| 364 | 47 | 300 | 5.00 |
| 366 | 39 | 302 | 5.03 |
| 368 | 33 | 304 | 5.07 |
| 370 | 27 | 306 | 5.10 |
| 372 | 21 | 308 | 5.13 |
| 374 | 15 | 310 | 5.17 |
| 376 | 11 | 312 | 5.20 |
| 378 | 8 | 314 | 5.23 |
| 380 | 5 | 316 | 5.27 |
| 382 | 4 | 318 | 5.30 |
| 384 | 3 | 320 | 5.33 |
| 386 | 4 | 322 | 5.37 |
| 388 | 3 | 324 | 5.40 |
| 390 | 3 | 326 | 5.43 |
| 392 | 3 | 328 | 5.47 |
| 394 | 3 | 330 | 5.50 |
| 396 | 3 | 332 | 5.53 |
| 398 | 3 | 334 | 5.57 |
| 400 | 3 | 336 | 5.60 |
| 402 | 3 | 338 | 5.63 |
| 404 | 3 | 340 | 5.67 |
| 406 | 2 | 342 | 5.70 |
| 408 | 2 | 344 | 5.73 |
| 410 | 1 | 346 | 5.77 |
| 412 | 2 | 348 | 5.80 |
| 414 | 1 | 350 | 5.83 |

### DESCRIPTION OF THE FIGURES

FIG. 1 Schematic overview of the arrangement of the power sources for the “power” firing process of the dry aerosol generator according to the invention

FIG. 1A: Test variant 1

FIG. 1B: Test variant 2A and 2B

FIG. 1C: Test variant 3A and 3B

FIG. 1D: Test variant 4

FIG. 2 Comparative measurements for demonstrating the effectiveness of the developed “intelligent power electronics (IPE)” in the dry aerosol generator

FIG. 3 Monitoring of radioactivity values during dry aerosol preparation in the generator

FIG. 4 Results of the measurement for monitoring the residual oxygen content when building up the inert gas-shielding gas atmosphere of the combustion chamber of the dry aerosol generator (resolution of the diagram “y in 10,000”)

FIG. 5 Results of the measurement for monitoring the residual oxygen content when building up the inert gas-shielding gas atmosphere of the combustion chamber of the dry aerosol generator (resolution of the diagram “y in 100”)

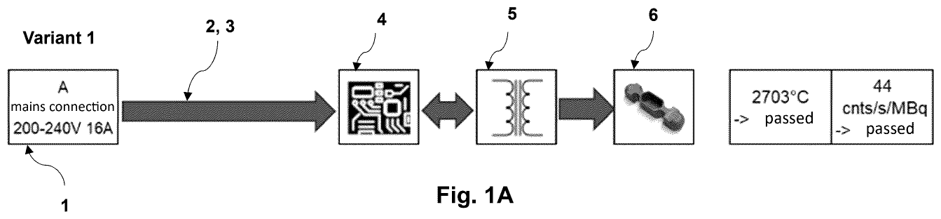

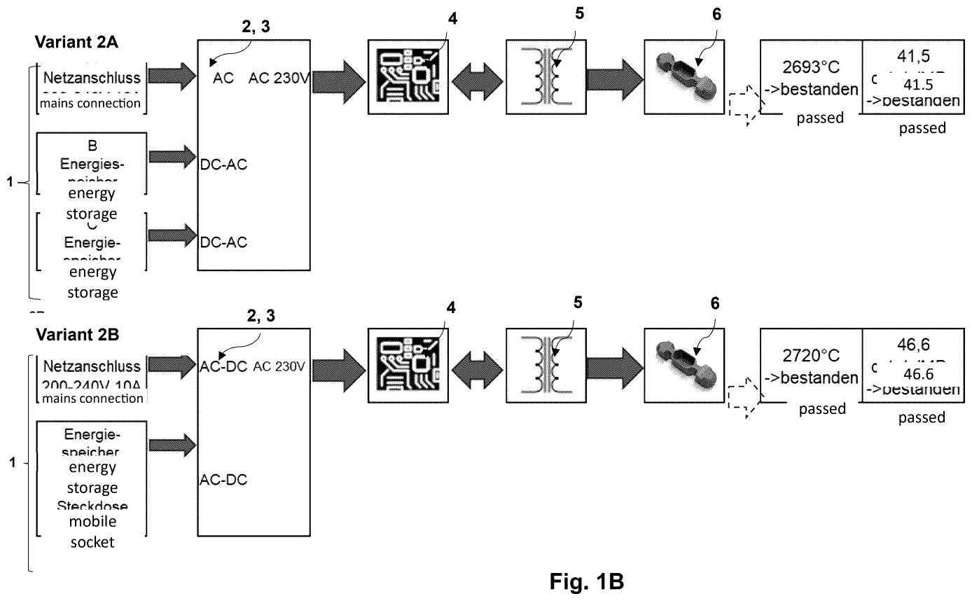

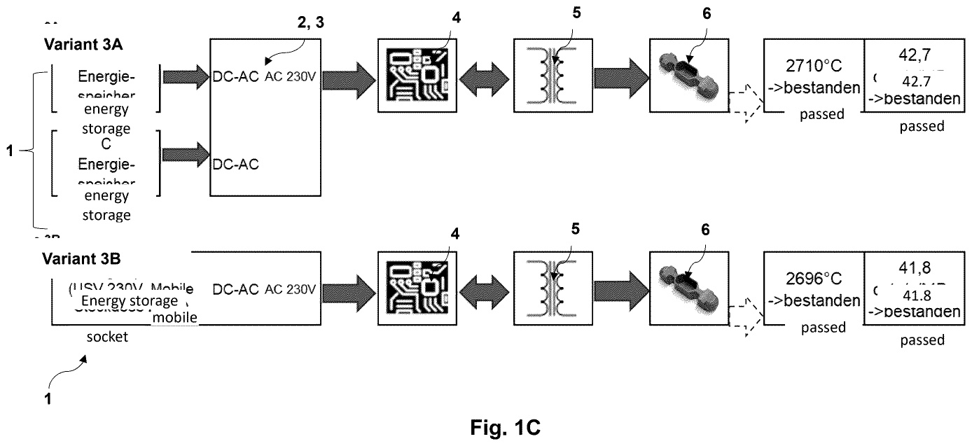

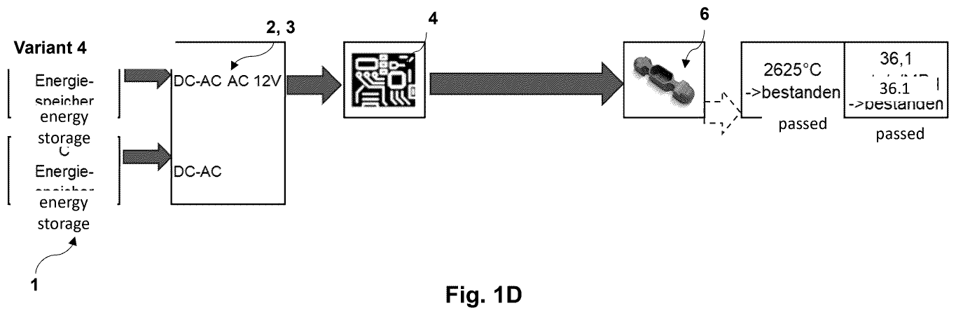

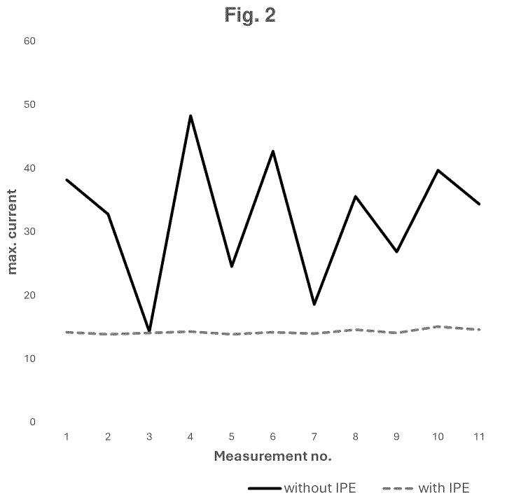

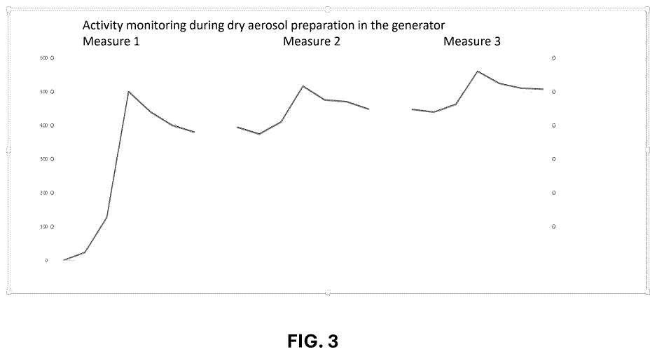

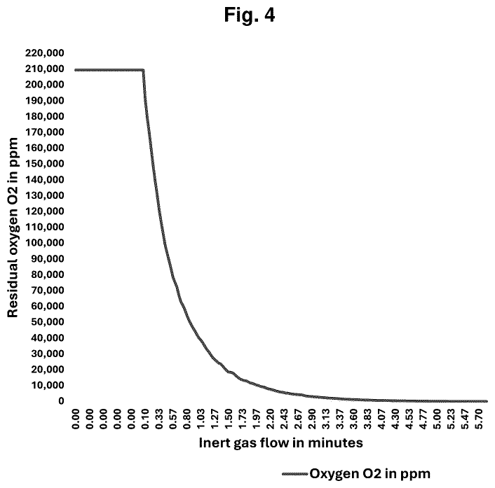

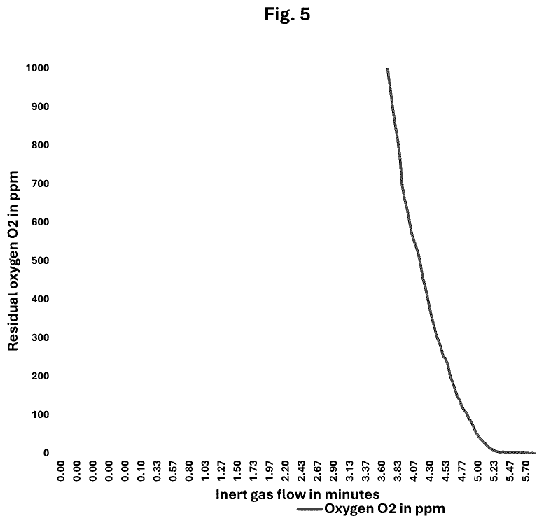

### REFERENCE SIGNS

1 Power source(s)
2 Power conditioning unit
3 Power source combination unit
4 Intelligent power electronics
5 Power transformer
6 Generator combustion chamber

1. An apparatus for producing a radioactively labeled dry aerosol comprising
a carbon crucible with an open cavity suitable for filling with radioactive nuclide,
a combustion chamber which is suitable for holding a carbon crucible,
a current pulse generator that enables flash heating of the carbon crucible and a radioactive nuclide contained therein to form a dry aerosol of radioactively labeled carbon particles, which can then be passed from the apparatus into a patient's lungs for the purpose of medical imaging,
a control unit,
wherein the apparatus is characterized in that
the filling volume of the carbon crucible is at least 0.1 mL-1 mL, and the apparatus further comprising: comprises the following:
a probe for measuring the oxygen content in the combustion chamber,
a vacuum pump for venting the combustion chamber,
an air compressor for ventilating the combustion chamber,
a camera that enables the filling of the carbon crucible to be monitored,
a gamma counter which is capable of measuring a change in the amount of radioactivity in the carbon crucible and/or the combustion chamber,
a flow meter that is configured to determine a patient's respiratory volume,
at least one battery and/or PowerCap/buffer capacitor and/or high-performance capacitor,
an inverter for transforming the direct current of a battery and/or PowerCap/buffer capacitor and/or high-performance capacitor into an alternating voltage sine wave,
an intelligent power electronics unit for power grid operation of the apparatus with transformer, which is configured to analyze the sine wave of the AC voltage of the grid current, wherein the sine wave preferably exhibits a standard frequency of 50-60 Hz and the AC voltage of the mains current is preferably 100 to 400 volts, preferably 110-260 volts, in particular 230 volts, and
an external radioactivity sensor connected to the apparatus and configured to detect the increase in radioactivity in the patient's lungs upon administration of the produced radioactive dry aerosol.

2. The apparatus according to claim 1, wherein the radioactive nuclide is selected from the group consisting of: technetium (Tc-99m), gallium-68, molybdenum-99, chromium-51, holmium-166, rubidium-82, iron-59, lutetium-177, palladium-103, potassium-42, scandium-47, selenium-75, sodium-24, xenon-133, ytterbium-169, ytterbium-177, iodine-131, iodine-125, samarium-153, rhenium-186, lutetium-177, phosphorus-32, caesium-131.

3. The apparatus according to claim 1, wherein the apparatus does not comprise interchangeable contacts which have to be replaced by the user.

4. The apparatus according to claim 1, wherein the radioactively labeled dry aerosol comprises nanoparticles.

5. The apparatus according to the claim 4, wherein at least 75% of the nanoparticles exhibit a diameter of less than 100 nm.

6. The apparatus according to claim 1, wherein the apparatus comprises means for automatically bringing the carbon crucible into contact with electrical contacts, wherein the means are preferably also configured for automatic “looping-in” of contact between the crucible and the electrical contacts.

7. Method A method for producing radioactively labeled dry aerosol comprising
providing an apparatus for producing radioactively labeled dry aerosol according to claim 1,
carrying out a breathing test (pre-breathing) on the patient, wherein the patient's respiratory volume is measured in order to assess their fitness for examination,
camera-monitored filling of a carbon crucible inserted into the combustion chamber of the apparatus with a radioactive nuclide,
evacuating the air from the combustion chamber to create a vacuum,
filling the combustion chamber with argon gas, with simultaneous monitoring of any residual oxygen present, to generate an inert argon, nitrogen or shielding gas atmosphere in the combustion chamber,
preheating the carbon crucible,
heating the filled carbon crucible to produce a radioactively labeled dry aerosol or nanoparticle composite, wherein the electricity required during heating is provided, either
by the at least one battery and/or PowerCap/buffer capacitor and/or high-performance capacitor contained therein, or
by mains current, which is supported and optionally amplified by the at least one battery and/or PowerCap/buffer capacitor and/or high-performance capacitor, or
by mains current, wherein the intelligent power electronics unit analyzes the sine wave of the AC voltage of the mains current and only switches on the transformer when the inrush load of the transformer reaches a predefined, favorable point in time on the sine wave of the mains current, such that the filled carbon crucible is heated to 2,500-3,000° C., in less than 2 seconds, preferably up to 1.8 seconds, preferably up to 1.5 seconds, preferably up to 1.2 seconds,
lung activity monitoring during inhalation by a patient of the produced radioactively labeled dry aerosol or nanoparticle composite, wherein the amount of radioactivity inhaled by the patient is monitored by a radioactivity sensor, preferably localized on the patient, and
purging of the combustion chamber with compressed air to remove the radioactive particles, wherein the radioactive particles are removed by a filter from the outflowing air, argon, nitrogen or shielding gas mixture, and optional monitoring of the change in radioactivity in the combustion chamber.

8. The method according to claim 7, wherein the radioactive nuclide is selected from the group consisting of: technetium (Tc-99m), gallium-68, molybdenum-99, chromium-51, holmium-166, rubidium-82, iron-59, lutetium-177, palladium-103, potassium-42, scandium-47, selenium-75, sodium-24, xenon-133, ytterbium-169, ytterbium-177, iodine-131, iodine-125, samarium-153, rhenium-186, lutetium-177, phosphorus-32, caesium-131.

9. The method according to claim 7, wherein during the heating of the filled carbon crucible the direct current of the at least one battery and/or PowerCap/buffer capacitor and/or high-performance capacitor is converted into an alternating voltage by means of pulse width modulation or an inverter, such that the voltage converted from the at least one battery and/or PowerCap/buffer capacitor and/or high-performance capacitor is switched to the sine wave of the alternating voltage of the mains current input of the apparatus and thus amplifies the power supply current to preferably ≥16 amperes.

10. The method according to claim 7, wherein during the breathing test the patient breathes through a tube set, such that when breathing in, filtered room air flows through a flow meter, which measures the inhaled volume per breath.

11. The apparatus according to claim 1, wherein the filling volume of the carbon crucible is between 0.3-0.6 mL.

12. The apparatus according to claim 1, further comprising an apparatus for integrating an argon or shielding gas source.

13. The apparatus according to claim 1, further comprising an external ventilation bag which can be connected to the apparatus and is configured to increase the volume of radioactively labeled dry aerosol inhaled by a patient.

14. The apparatus according to claim 2, wherein the radioactive nuclide is technetium (Tc-99m) or gallium 68.

15. The apparatus according to claim 4, wherein the nanoparticles preferably exhibit an average diameter between 5-250 nm.

16. The method according to claim 7, further comprising a pressure test to check the seal of the combustion chamber.

17. The method according to claim 7, further comprising vaporization of a carrier liquid of the radioactive nuclide.

18. The method according to claim 7, further comprising drying of the radioactive nuclide.

19. The method according to claim 7, wherein the carbon crucible is preheated to 500° C.

20. The method according to claim 7, wherein the filled carbon crucible is heated to 2,750° C.
